(19) United States
(12) Patent Application Publication
(10) Pub. No.: US 2025/0145924 A1
(43) Pub. Date: May 8, 2025

(54) CELL DEACTIVATION DEVICE

(71) Applicant: Georgia Tech Research Corporation, Atlanta, GA (US)

(72) Inventors: Todd Sulchek, Atlanta, GA (US); Eric Dixon, Morrisville, NC (US); Peter Shankles, Atlanta, GA (US)

(21) Appl. No.: 18/832,684

(22) PCT Filed: Jan. 25, 2023
(86) PCT No.: PCT/US2023/061263

§ 371 Date: Jul. 24, 2024

Related U.S. Application Data
(60) Provisional application No. 63/303,157, filed on Jan. 26, 2022.
(60) Provisional application No. 63/315,300, filed on Mar. 1, 2022.

(51) Int. Cl. C12M 3/06; C12M 1/00; C12M 1/42

(52) U.S. Cl. CPC C12M 23/16; C12M 23/38; C12M 23/40; C12M 35/02

(57) ABSTRACT

A cell deactivation device includes a cell deactivation container. The cell deactivation container includes a plurality of microfluidic channels, a lid covering the plurality of microfluidic channels, the lid having a thickness of less than about 150 microns, and a distribution manifold configured to distribute a fluid to the plurality of microfluidic channels. The cell deactivation device further includes an irradiation source configured to generate an electron beam and transmit the electron beam through the lid of the cell deactivation container and into a fluid passing through the plurality of microfluidic channels.

### CROSS-REFERENCE TO RELATED APPLICATIONS

This application claims the benefit of U.S. Provisional Patent Application No. 63/303,157 filed Jan. 26, 2022 entitled “Microfluidics Enabled Sterilization of Complex Drug Products as Continuous End-Line Process”, and U.S. Provisional Patent Application No. 63/315,300 filed Mar. 1, 2022 entitled “System of Cell Deactivation”, each of which is incorporated by reference herein in its entirety.

### SUMMARY

In some embodiments, there is a cell deactivation device including a cell deactivation container, the cell deactivation container including a plurality of microfluidic channels, a lid covering the plurality of microfluidic channels, the lid having a thickness of less than about 150 microns, and a distribution manifold configured to distribute a fluid to the plurality of microfluidic channels. The cell deactivation device further includes an irradiation source configured to generate an electron beam and transmit the electron beam through the lid of the cell deactivation container and into a fluid passing through the plurality of microfluidic channels. In some embodiments, the electron beam has an energy of less than 100 keV. In some embodiments, the distribution manifold include a plurality of fluid distribution channels in fluid communication with one another and including one or more rounded edges where adjacent fluid distribution channels fluidly connect to one another.

In some embodiments, the lid has a thickness of between about 25 microns to about 150 microns. In some embodiments, the lid has a thickness of between about 50 microns to about 150 microns. In some embodiments, the lid has a thickness of between about 75 microns to about 150 microns. In some embodiments, the lid has a thickness of between about 100 microns to about 150 microns.

In some embodiments, each of the plurality of microfluid channels has a maximum width and a maximum depth, wherein an aspect ratio of the maximum width to the maximum depth of each of the plurality of microfluidic channels is less than about 5:1. In some embodiments, each of the plurality of microfluidic channels has a maximum width and a maximum depth, wherein an aspect ratio of the maximum width to the maximum depth of each of the plurality of microfluidic channels is less than about 10:1. In some embodiments, each of the plurality of microfluidic channels has a maximum width and a maximum depth, wherein an aspect ratio of the maximum width to the maximum depth of each of the plurality of microfluidic channels is between about 1:1 to about 10:1. In some embodiments, each of the plurality of microfluidic channels has a maximum width and a maximum depth, wherein an aspect ratio of the maximum width to the maximum depth of each of the plurality of microfluidic channels is from about 2.5:1 to about 7.5:1.

In some embodiments, each of the plurality of microfluidic channels has a maximum width and a maximum depth, wherein an aspect ratio of the maximum width to the maximum depth of each of the plurality of microfluidic channels is from about 4:1 to about 6:1. In some embodiments, each of the plurality of microfluidic channels has a maximum depth, wherein the sum of the maximum depth and the thickness of the lid is no greater than about 250 microns. In some embodiments, each of the plurality of microfluidic channels has a maximum depth, wherein the sum of the maximum depth and the thickness of the lid is no greater than about 200 microns. In some embodiments, each of the plurality of microfluidic channels has a maximum width of between about 0.5 millimeters to about 5.0 millimeters. In some embodiments, each of the plurality of microfluidic channels has a maximum width of between about 1.0 millimeters to about 2.0 millimeters. In some embodiments, the lid comprises a metal and/or a plastic configured to allow a transmitted beam of electrons to pass from an irradiation source to a fluid flowing through the plurality of microfluidic channels.

In some embodiments, the cell deactivation container further comprises a distribution manifold configured to distribute a fluid to the plurality of microfluidic channels. In some embodiments, the distribution manifold configured to distribute a fluid to the plurality of microfluidic channels at a substantially equal volumetric flow rate. In some embodiments, the distribution manifold configured to distribute a fluid to the plurality of microfluidic channels, such that the volumetric flow rate of fluid through the plurality of microfluidic channels does not vary by more than about 25%. In some embodiments, the distribution manifold is configured to distribute a fluid to the plurality of microfluidic channels, such that the volumetric flow rate of fluid through the plurality of microfluidic channels does not vary by more than about 10%. In some embodiments, the distribution manifold is configured to distribute a fluid to the plurality of microfluidic channels, such that the volumetric flow rate of fluid through the plurality of microfluidic channels does not vary by more than about 5%. In some embodiments, the distribution manifold is configured to distribute a fluid to the plurality of microfluidic channels, such that the volumetric flow rate of fluid through the plurality of microfluidic channels does not vary by more than about 1%.

In some embodiments, the distribution manifold has a maximum depth and wherein each of the plurality of microfluidic channels have a maximum depth, wherein an aspect ratio of the maximum depth of the distribution manifold to the maximum depth of the plurality of microfluidic channels is between about 3:1 to about 50:1. In some embodiments, the aspect ratio of the maximum depth of the distribution manifold to the maximum depth of the plurality of microfluidic channels is at least about 7:1. In some embodiments, the aspect ratio of the maximum depth of the distribution manifold to the maximum depth of the plurality of microfluidic channels is at least about 10:1. In some embodiments, the aspect ratio of the maximum depth of the distribution manifold to the maximum depth of the plurality of microfluidic channels is at least about 15:1. In some embodiments, the aspect ratio of the maximum depth of the distribution manifold to the maximum depth of the plurality of microfluidic channels is at least about 25:1.

In some embodiments, the irradiation source is configured to generate an electron beam and transmit the electron beam through the lid of the cell deactivation container and into a fluid passing through the plurality of microfluidic channels to inhibit replication of cells included in the fluid and render the cells replication-incompetent. In some embodiments, the fluid contained within the microfluidic channels includes one or more pathogens, and the irradiation source is configured to generate an electron beam and transmit the electron beam through the lid of the cell deactivation container and into a fluid passing through the plurality of microfluidic channels to deactivate the one or more pathogens. In some embodiments, the one or more pathogens include at least one of a bacterium, a virus, a fungus, or a parasite. In some embodiments, the one or more pathogens comprise nucleic acid molecules comprising DNA and/or RNA. In some embodiments, the deactivation of the one or more pathogens alters gene expression of the one or more pathogens.

### BACKGROUND

The present invention generally relates to a cell deactivation device and, more particularly, to containers for use in a cell deactivation device using low energy electron irradiation.

### BRIEF DESCRIPTION OF THE SEVERAL VIEWS OF THE DRAWINGS

The foregoing summary, as well as following detailed description of embodiments of the cell deactivation device, will be better understood when read in conjunction with the appended drawings of exemplary embodiments. It should be understood, however, that the invention is not limited to the precise arrangements and instrumentalities shown.

In the drawings:

FIG. 1 is a schematic illustration of a cell deactivation device for deactivating cells using low energy electron irradiation in accordance with an exemplary embodiment of the present invention;

FIG. 2 is a graph showing the electron penetration dose percentage of maximum dose vs the depth in water for various electron source voltage levels;

FIGS. 3A-3C depict top cross-sectional views of various embodiments of cell deactivation containers in accordance with embodiments of the present disclosure;

FIGS. 4A-4C depict the velocity magnitude of liquids passing through the cell deactivation containers shown in FIGS. 3A-3C.

FIG. 5 illustrates altering the channel size of a distribution layer and the corresponding velocity magnitude distributions;

FIG. 6A illustrates test simulation results of the velocity magnitude of a fluid passing through a cell deactivation container;

FIG. 6B illustrates another test simulation result of the velocity magnitude of fluid passing through the cell deactivation container of FIG. 6A;

FIG. 6C is a line graph illustrating various velocity magnitudes of liquids flowing through the cell deactivation container of FIG. 6A;

FIGS. 7A-7B illustrates test results of the flow rate of liquids passing through microfluidic channels of different dimensions are shown;

FIGS. 8A-8B are side cross-sectional illustrations of cell deactivation containers used in rapid prototyping fabrication processes;

FIG. 9A is a representation of relation between apparent dose and beam current;

FIG. 9B illustrates a test simulation of the shear rate of fluids passing through the first distribution layer of the cell deactivation container of FIG. 6A;

FIGS. 10A-10C illustrate an embodiment of a cell deactivation container in accordance with an exemplary embodiment of the present disclosure. FIG. 10A is a top elevational view thereof and FIG. 10B is a side elevational view thereof. FIG. 10C shows the cell deactivation container of FIG. 10A with a fluid contained partially therein and a microscope slide positioned over a top surface of the container;

FIG. 11 is a magnified view of cells within a channel of the exposure layer of the cell deactivation container shown in FIGS. 10A-10C;

FIG. 12 is a graphical representation of the measured average cell velocity of cells within the channel shown in FIG. 11;

FIG. 13 is a graph illustrating the penetration depth model for various two laminate materials;

FIG. 14 is a schematic illustration of an electron beam passed through an aperture at a cell deactivation container;

FIG. 15A is a magnified view of a channel containing cells included at the start of an HEK 293 cell culture during a controlled test;

FIGS. 15B-15C depict processing results of the HEK 293 culture from a syringe and from the 2 L/hr sample;

FIGS. 15D-15E are bar graphs depicting the viability and cell concentration after processing the HEK 293 culture for the various testing platforms;

FIG. 16A is a perspective view of a cell deactivation container in accordance with an embodiment of the present disclosure;

FIG. 16B is a magnified view of a portion of the cell deactivation container shown in FIG. 16A

FIGS. 17A-17D are illustrations of observed test results from processing high cell concentrations; and

FIG. 18 is an illustration of an instance of cells entering a distribution layer of the cell deactivation container shown in FIG. 16A.

### DETAILED DESCRIPTION OF EXEMPLARY EMBODIMENTS OF THE INVENTION

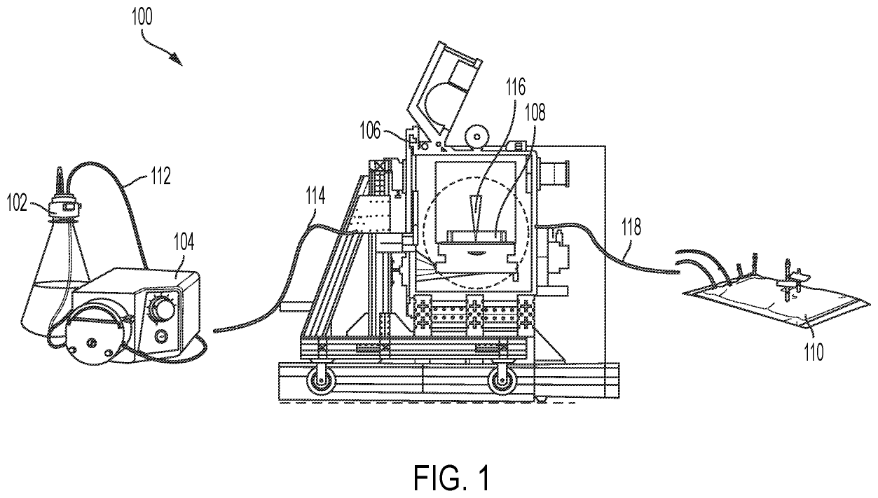

Referring to the drawings in detail, wherein like reference numerals indicate like elements throughout, there is shown in FIGS. 1-18 a system for cell deactivation in accordance with exemplary embodiments of the present invention.

Referring to FIG. 1, there is shown a schematic illustration of a cell deactivation device, generally designated 100 and referred to as device 100 for short, for cell deactivation in accordance with an exemplary embodiment of the present disclosure. Cell deactivation may refer to rendering one or more cells replication-incompetent such that the cells are not capable of replicating. In some embodiments, the device 100 is configured to expose one or more cells to low energy electron irradiation such that the cells remain biologically active while not replication competent. In this manner, the device 100 may be configured for the production of inactivated vaccines. In other embodiments, the device 100 is configured to biologically deactivate one or more cells such that the device 100 produces a sterilized biological sample. In some embodiments, the cell deactivation device 100 may alternatively be referred to as a system for cell deactivation.

The device 100 may be configured to receive a fluid containing one or more cells for which cell deactivation is desired. For example, the fluid may contain one or more pathogens (e.g., bacteria, virus, or other microorganisms that may cause disease) for which inactivation is desired. In some embodiments, the device 100 is configured to deactivate one or more advantageous pathogens (e.g., bacterium, viruses, fungi, parasites or other microorganisms that may cause disease) from fluids or vaccines. In some embodiments, the one or more pathogens may comprise nucleic acid molecules comprising DNA and/or RNA. In some embodiments, the device 100 is configured to render the cells replication incompetent. In some embodiments, the deactivation of the one or more pathogens may alter the gene expression of the one or more pathogens. In some embodiments, the device 100 includes a fluid source 102, a fluid pump 104, an irradiation source 106, a cell deactivation container 108, and a fluid reservoir 110. The fluid source 102 may be any suitable container for containing a fluid for which cell deactivation is desired. The fluid source 102 may be in fluid communication with the fluid pump 104 via a first fluid conduit 112. In some embodiments, the first fluid conduit 112 may be a tube or pipe that fluidly couples the fluid source 102 to the fluid pump 104 such that fluid from the fluid source 102 may flow through the first fluid conduit 112 and into the fluid pump 104. The fluid pump 104 may be in fluid communication with the cell deactivation container 108 via a second fluid conduit 114. The second fluid conduit 114 may be a tube or pipe that fluidly couples the fluid pump 104 to the cell deactivation container 108 such that fluid may be transferred from the fluid pump 104 through the second fluid conduit 114 and into the cell deactivation container 108. In this manner, the fluid pump 104 may transfer fluid from the fluid source 102 to the cell deactivation container 108. The fluid pump 104 may be any pump suitable for transferring fluid from the fluid source 102 to the cell deactivation container 108 at a predetermined flow rate. In some embodiments, the fluid pump 104 may be a positive displacement pump, such as, for example, a peristaltic pump.

In some embodiments, the irradiation source 106 may include a housing defining a chamber within which the cell deactivation container may be positioned. The irradiation source 106 may include one or more electron beam generators (not shown) each configured to generate an electron beam 116 and transmit it into the fluid passing through the cell deactivation container. In this manner, the irradiation source 106 may be configured to expose fluid contained within the cell deactivation container 108 to low energy electron irradiation (LEEI) to deactivate cells included in the fluid. In some embodiments, the irradiation source 106 may include radiation shielding configured to prevent radiation from exiting the chamber that the cell deactivation container 108 is positioned within. Because the irradiation source 106 is configured to generate an LEEI beam, the radiation shielding requirements of the irradiation source 106 may be reduced.

In some embodiments, the cell deactivation container 108 is in fluid communication with the fluid reservoir 110 such that the fluid exposed to the LEEI may be transferred from the cell deactivation container and to the fluid reservoir 110. The fluid reservoir 110 may be any suitable storage device or container for containing the fluid containing the deactivated cells. In some embodiments, there is a third fluid conduit 118 that fluidly couples the cell deactivation container 108 to the fluid reservoir 110. In some embodiments, the fluid reservoir 110 may be detachable from the third fluid conduit 118 such that the fluid reservoir 110 may be detached and replaced with a different fluid reservoir.

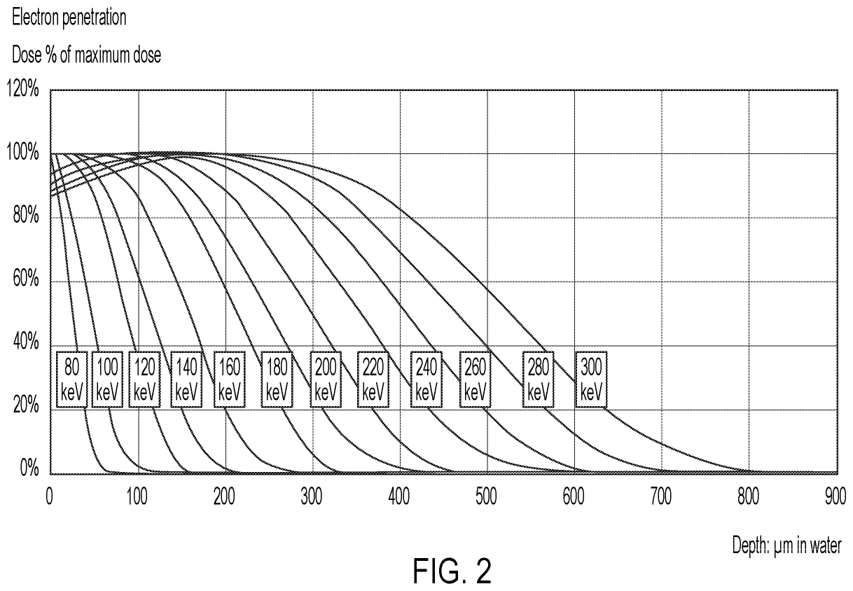

Referring to FIG. 2, there is shown a graph illustrating the electron penetration of electron beams at various electron voltages based on the dose percentage of a maximum dose and the depth in water. As illustrated, electron beams generated at voltages ranging from 80 kiloelectron volts (keV) to 300 keV are shown. The x-axis of the graph represents depth in microns in water and ranges from 0 to 900 microns. The y-axis of the graph represents the dose percentage of maximum dose and ranges from 0% to 120%. As shown in the graph there is less than an 80% dose of maximum dose at 200 micron depth for a 200 keV electron beam. In some embodiments, the device 100 is configured to generate an electron beam at a voltage between about 85 keV to about 300 keV. In some embodiments, the voltage of the electron beam is dependent upon the desired penetration depth. As illustrated in FIG. 2, the voltage of the electron beam and penetration depth are directly related (e.g., increasing voltage results in increased penetration depth). In some instances, a penetration depth of about 200 microns may be preferred. In some instances, electron beams generated at lower voltages (e.g., about 85 keV) may result in a “braking radiation” which refers to electromagnetic radiation produces by the deceleration of a charged particle (e.g., an electron) passing through matter in the vicinity of a strong electric field of an atomic nuclei. Electron beams generated at lower voltages may require less radiation shielding than electron beams generated at higher voltages.

As shown in FIG. 2, the penetration depth of the electron beams is relatively low and therefore, it may be desirable to provide the liquid for which LEEI is desired in a similarly thin layer. In some embodiments, the cell deactivation container 108 is configured to provide the liquid for which LEEI is desired in a low fluid thickness such that electrons may penetrate through at least a substantial portion of the fluid thickness to effect cell deactivation.

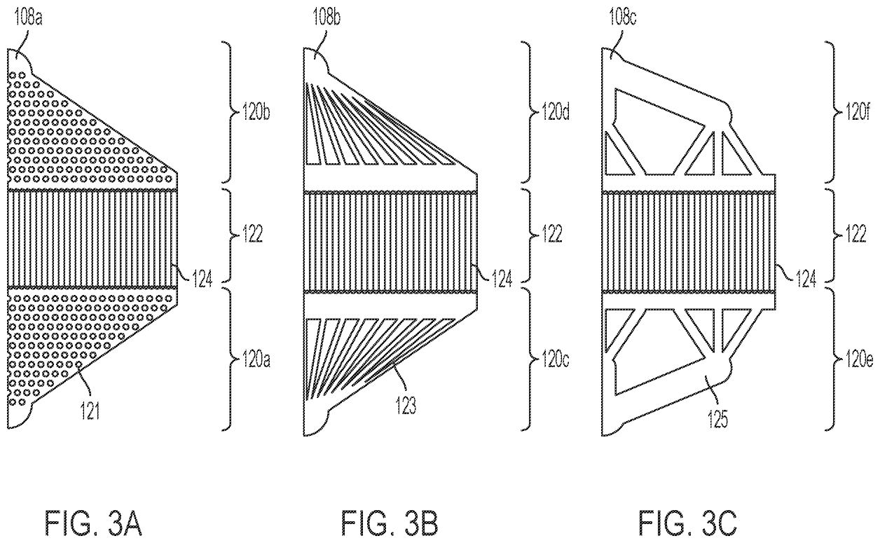

Referring to FIGS. 3A-3C, there is shown various embodiments of cell deactivation containers in accordance with embodiments of the present disclosure. In FIG. 3A, there is shown a first embodiment of a cell deactivation container 108a. The cell deactivation container 108a may include a first distribution layer 120a, an exposure layer 122, and a second distribution layer 120b. The first distribution layer 120a may be in fluid communication with the exposure layer 122 such that fluid may flow through the first distribution layer 120a and into the exposure layer 122. The exposure layer 122 may be in fluid communication with the second distribution layer 120b such that fluid may flow from the exposure layer 122 and to the second distribution layer 120b. Each of the distribution layers 120a, 120b may include one or more pillars 121 configured to control the flow rate of the liquid passing through the respective distribution layer 120a, 120b. The exposure layer 122 may define one or more microfluidic channels 124, referred to as channels 124 herein for short, extending from the first distribution layer 120a to the second distribution layer 120b.

In some embodiments, fluid may be received from the fluid pump 104 at the first distribution layer 120a, and flow through the first distribution layer 120a to the exposure layer 122. The fluid may proceed to flow through the channels 124 of the exposure layer 122 where the fluid is exposed to LEEI thereby generating a fluid containing deactivated cells. The fluid containing deactivated cells may flow from the exposure layer 122, through the second distribution layer 120b and out to the fluid reservoir 110. In this manner, the distribution layers 120a and/or 120b may act as a distribution manifold configured to distribute the fluid to and/or receive fluid from the plurality of microfluidic channels 124. In some embodiments, the distribution layers 120a, 120b may be a mirror image of one another. In some embodiments, the distribution manifold may be configured to distribute a fluid to the plurality of channels 124 at a substantially equal volumetric flow rate. For example, the arrangement of the pillars 121 within the first distribution layer 120a may cause a fluid flowing through the first distribution layer 120a and into the channels 124 to enter each of the channels 124 at substantially the same volumetric flow rate. In some embodiments, the distribution manifold may be configured to distribute a fluid to the plurality of channels 124 such that the volumetric flow rate of fluid flowing through the plurality of channels 124 does not vary by more than about 25%, about 10%, about 5% or about 1%.

Referring to FIG. 3B, there is shown a second embodiment of a cell deactivation container 108b. The cell deactivation container 108b may be generally the same as cell deactivation container 108a except that the first and second distribution layers 120c, 120d are different. For example, instead of pillars 121 (as shown in FIG. 3A), the distribution layers 120c, 120d may include one or more fluid distribution channels 123. The fluid distribution channels 123 may be arranged in a generally spoke-like formation as shown in FIG. 3B. Fluid may flow through the fluid distribution channels 123 and into the exposure layer 122. It will be understood that the position, size and number of fluid distribution channels 123 may be altered in order to alter the flow rate of fluid flowing through the distribution layers 120c, 120d. In some embodiments, the fluid distribution channels 123 have generally the same width.

Referring to FIG. 3C, there is shown a third embodiment of a cell deactivation container 108c. The cell deactivation container 108c may be generally the same as cell deactivation container 108a except that the first and second distribution layers 120e, 120f are different. For example, instead of pillars 121 (as shown in FIGS. 3A), the distribution layers 120e, 120f may include one or more fluid distribution channels 125. The fluid distribution channels 125 may be arranged in a generally branching formation as shown in FIG. 3C. The fluid distribution channels 125 may be configured to alter the flow rate of fluid flowing through the first and second distribution layers 120e, 120f. In some embodiments, one or more of the fluid distribution channels 125 have a width that is different than the width of one or more other fluid distribution channels 125. It will be understood that the position, size, and number of fluid distribution channels 125 may be altered in order to alter the flow rate of fluid flowing through the distribution layers 120e, 120f.

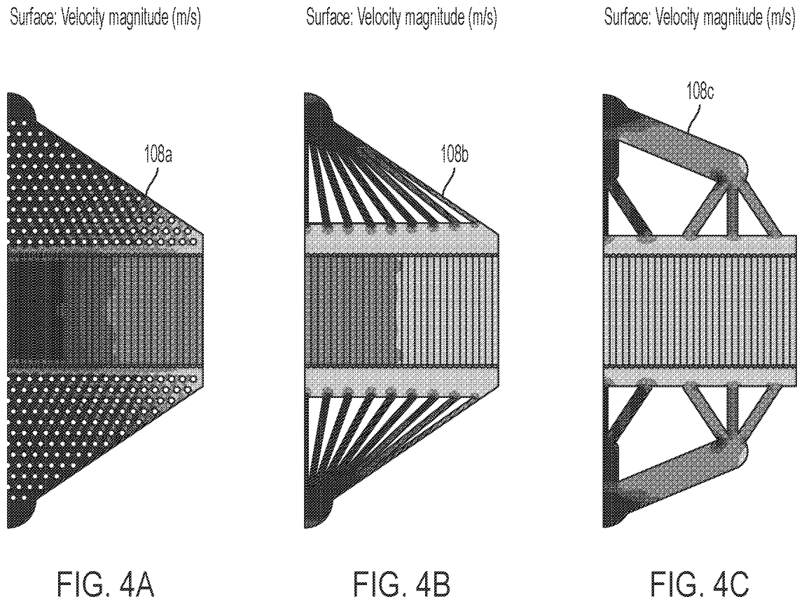

Referring to FIGS. 4A-4C, there is shown test simulation results of the flow rate of fluids passing through the cell deactivation containers 108a-108c of FIGS. 3A-3C. In FIGS. 4A-4C, the stippled areas indicate flow rates where the more densely stippled areas indicate higher flow rates than the more sparsely stippled areas. As shown, the flow rate of liquid flowing through the exposure areas is more evenly distributed in the cell deactivation container 108c than in either of the cell deactivation containers 108a, 108b. It may be beneficial to provide a generally even flow distribution within the exposure area to ensure a generally equal distribution of cell deactivation caused by exposure to LEEI. Furthermore, it may be beneficial to provide a more evenly distributed flow through the distribution layers to ensure that the flow of liquid passing through the exposure layer is also evenly distributed. In some embodiments, by providing a generally even flow distribution, clumping of cells contained within the fluid passing through the cell deactivation container may be broken up, thereby preventing clogging within the channels 121, 123, 125 and/or within the exposure layer 122. In some embodiments, the size, position, and/or arrangement of fluid distribution channels within the distribution layers may be altered.

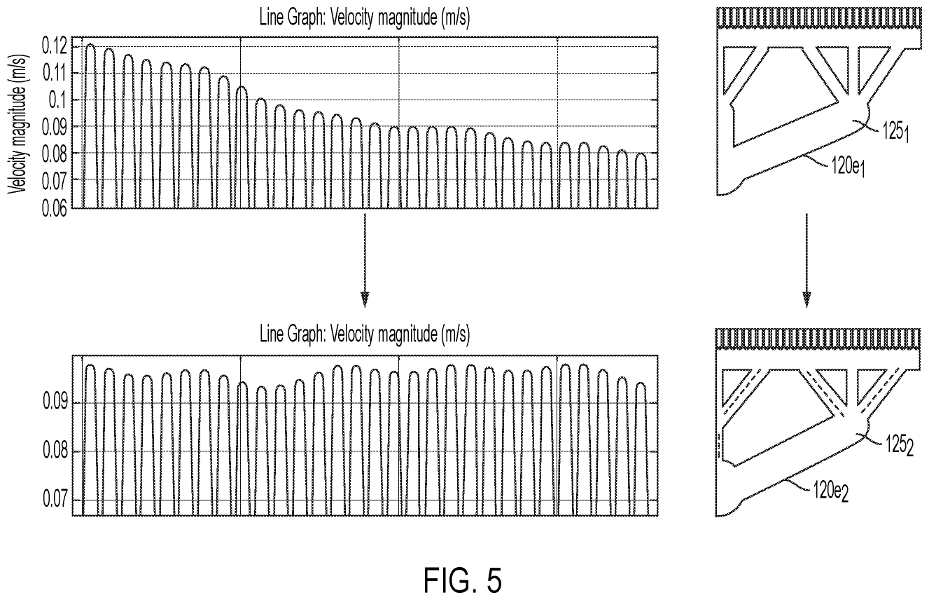

For example, referring to FIG. 5, there is shown an example of altering the size of one or more of the fluid distribution channels 125 of the cell deactivation container 108c in order to more evenly distribute the flow rate of fluid flowing through the first distribution layer 120e. In FIG. 5, the first distribution layer 120e₁ and fluid distribution channels 125₁ shown at the left of the page are generally the same as what is shown in FIG. 3C. The graph immediately below the fluid distribution layer 120e₁ illustrates the velocity magnitude of liquid flowing through the fluid distribution layer 120e₁ at different positions. As shown, the velocity magnitude varies from 0.08 m/s to about 0.12 m/s. As such, the size of one or more of the fluid distribution channels 125₁ is altered as shown in the fluid distribution layer 120e₂ on the right side of the page. As shown, the widths of some of the channels is increased and others are decreased resulting in fluid distribution channels 125₂. The channels 125₂ that were altered are illustrated as including a dotted line extending along the interior of the respective channel 125₂. The graph immediately below the fluid distribution layer 120e₂ illustrates the velocity magnitude of liquid flowing through the fluid distribution layer 120e₂. As shown, the variation in the velocity magnitude is reduced when compared to the velocity magnitude corresponding to fluid distribution layer 120e₁. In some embodiments, by altering the size of the fluid distribution channels 125₂, the variation in velocity magnitude is reduced to less than about 5%.

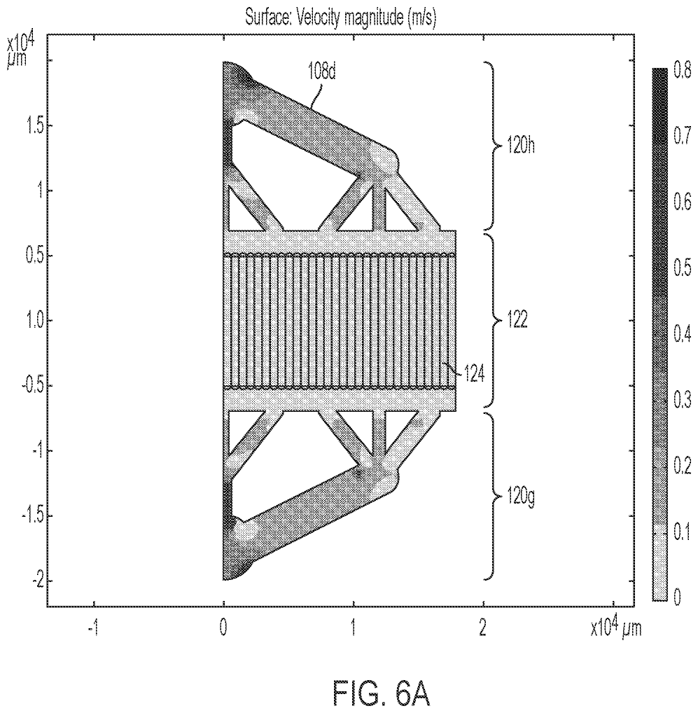

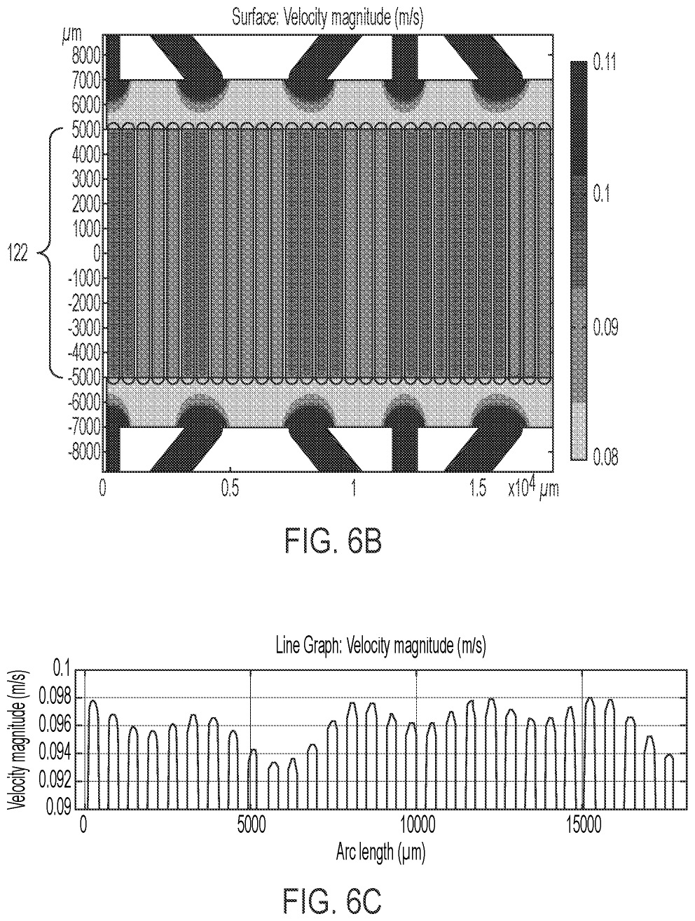

Referring to FIG. 6A there is shown test simulation results of the velocity magnitude of fluids passing through a cell deactivation container 108d. The cell deactivation container 108d is generally the same as cell deactivation container 108c except that the size of the fluid distribution channels 127 of the first and second distribution layers 120g, 120h have been altered similarly to what is depicted in FIG. 5. As shown, the velocity magnitude of fluid flowing through the cell deactivation container 108d is more evenly distributed than the velocity magnitude of fluid flowing through the cell deactivation containers 108a-108c (shown in FIGS. 4A-4C). Referring to FIG. 6B, there is shown another test simulation results of the velocity magnitude of fluids passing through cell deactivation container 108d. FIG. 6C is a line graph illustrating the various velocity magnitudes of liquids flowing through the cell deactivation container 108d. As can be seen in FIGS. 6A-6C, the distribution channels 120g, 120h provide a more evenly distributed, or balanced, flow rate across the channels 124 defined by the exposure layer 122.

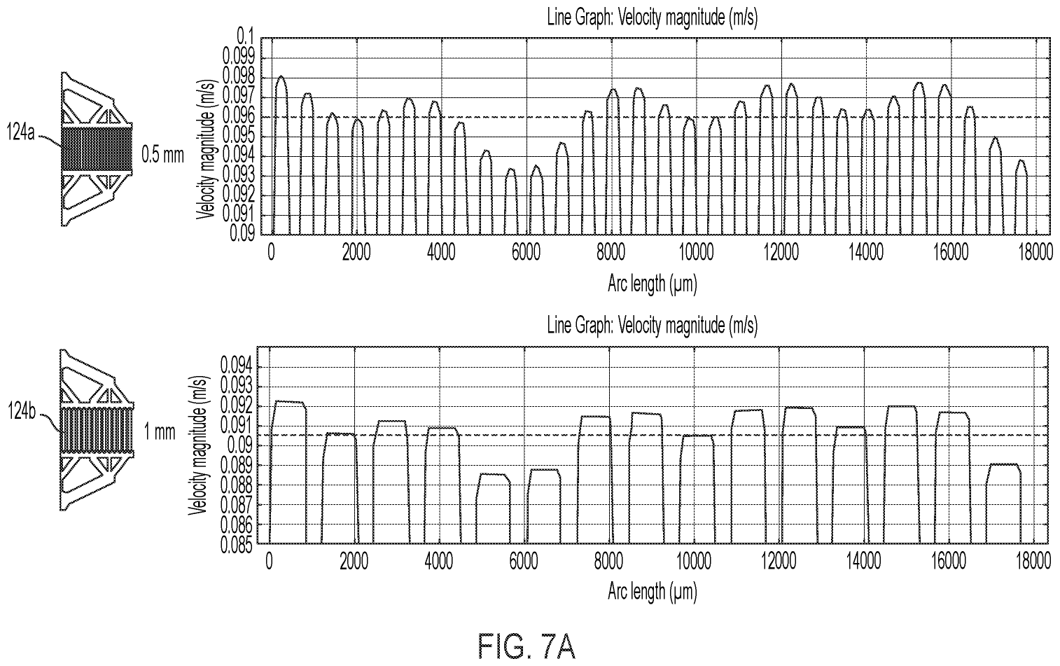

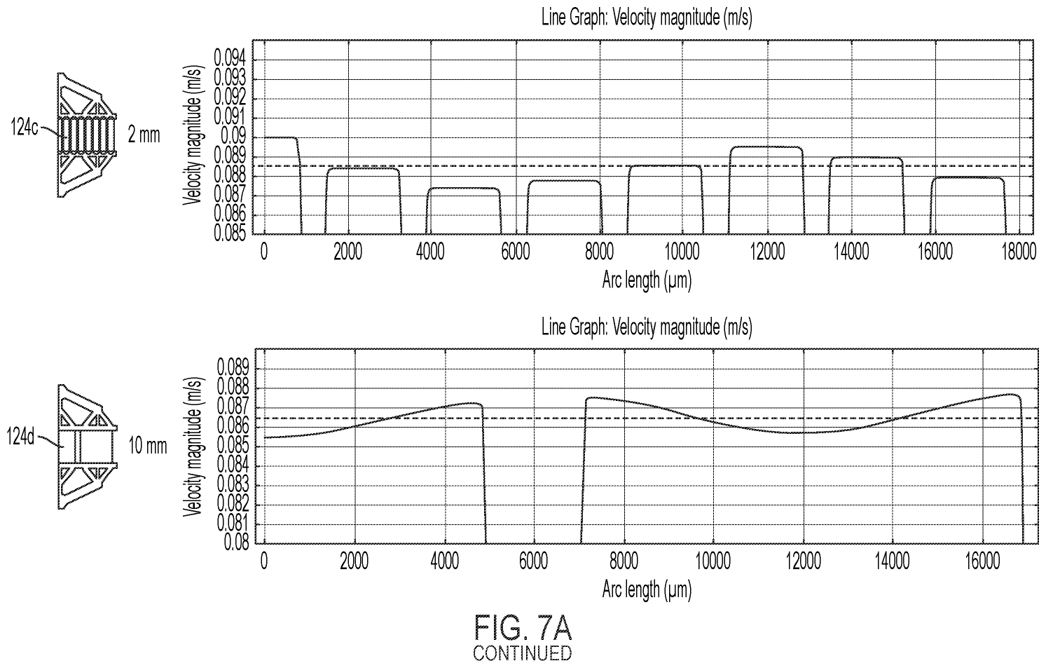

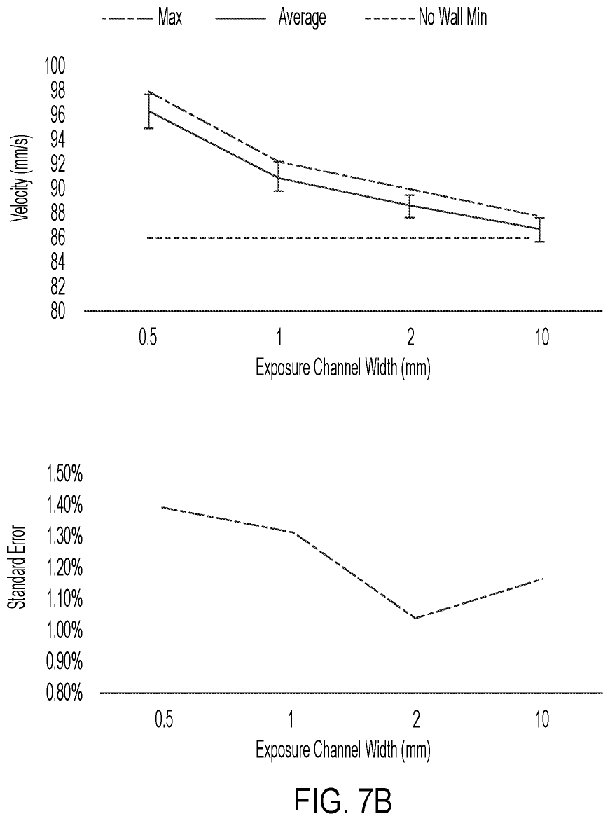

In some embodiments, the width of the channels 124 included in the exposure area 122 of any one of the cell deactivation containers 108a-108d may be altered to alter the flow rate of liquid flowing through said channels 124. For example, as shown in FIGS. 7A-7B, test results of the flow rate of liquids passing through channels 124a-124d where each of channels 124a-124d have a width that is different from each other are shown. As can be seen, as the width of the channels 124a-124d increases, the average velocity magnitude and variation in velocity magnitude decreases.

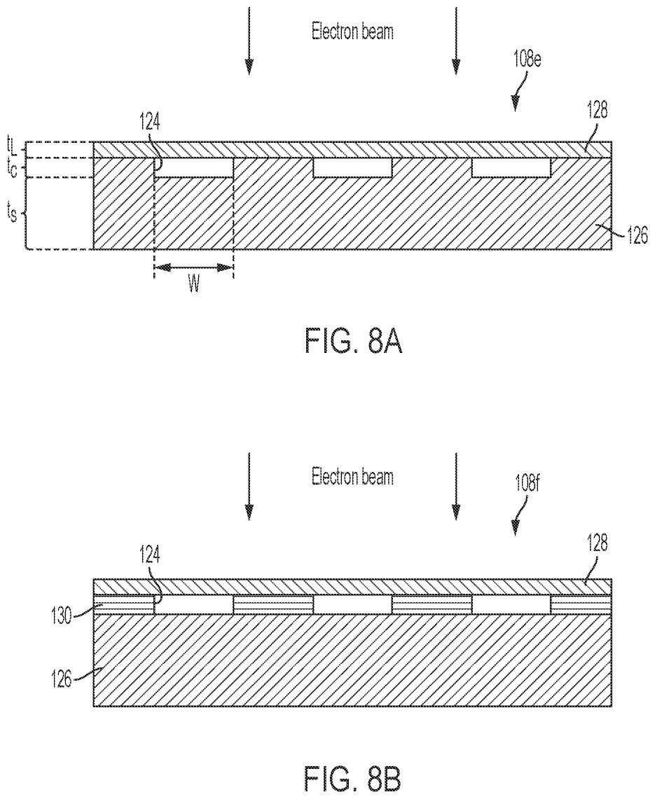

Referring to FIGS. 8A-8B, there is shown side cross-sectional illustrations of cell deactivation containers used in rapid prototyping fabrication processes. In FIG. 8A, the cell deactivation container 108e includes a substrate 126, one or more channels 124, and a lid 128. The substrate 126 may have a thickness tₛ that is greater than or equal to 500 microns, the channels 124 may have a thickness t_{c} of about 100 microns, and the lid 128 may have a thickness tₗ of about 50 microns. In some embodiments the thickness tₗ of the lid 128 is less than about 150 microns. In some embodiments, the thickness tₗ of the lid 128 is between about 25 microns to about 150 microns. In some embodiments, the thickness tₗ of the lid 128 is between about 50 microns to about 150 microns. In some embodiments, the thickness tₗ of the lid 128 is between about 75 microns to about 150 microns. In some embodiments, the thickness tₗ of the lid 128 is between about 100 microns to about 150 microns. In some embodiments, the thickness tₗ of the lid 128 is between about 75 microns to about 125 microns.

In some embodiments, the width W of the channel 124 may be about 2 millimeters. In some embodiments, the width W represents the maximum width of the channel 124 and the thickness t_{c} represents the maximum depth of the channel 124. In some embodiments, each channel 124 of the plurality of channels 124 included in any one of the cell deactivation containers 108 described herein have generally the same width W and thickness t_{c} as one another. In some embodiments, the maximum width of each of the channels 124 is between about 0.5 millimeters to about 5.0 millimeters. In some embodiments, the maximum width of each of the channels 124 is between about 1.0 millimeters to about 2.0 millimeters.

In some embodiments, the width to depth ratio (e.g., the width W to thickness t_{c}) of the channel 124 may be about 20:1. In some embodiments, by providing channels 124, having an aspect ratio of about 20:1, clumping of cells contained within a fluid flowing through the channels 124 may be broken up such that the cells are not clumped together. In some embodiments, the aspect ratio of the maximum width to the maximum depth of each of the plurality of channels 124 is less than about 5:1. In some embodiments, the aspect ratio of the maximum width to the maximum depth of each of the plurality of channels 124 is less than about 10:1. In some embodiments, the aspect ratio of the maximum width to the maximum depth of each of the plurality of channels 124 is between about 1:1 to about 10:1. In some embodiments, the aspect ratio of the maximum width to the maximum depth of each of the plurality of channels 124 is between about 2.5:1 to about 7.5:1. In some embodiments, the aspect ratio of the maximum width to the maximum depth of each of the plurality of channels 124 is between about 4:1 to about 6:1.

In some embodiments, the sum of the maximum depth of the channels 124 (e.g., the thickness t_{c} of the channels 124) and the thickness tₗ of the lid 128 is less than or equal to 250 microns. For example, the sum of the thicknesses t_{c} and tₗ may be less than or equal to 250 microns. In some embodiments, the sum of the maximum depth of the channels 124 and the thickness tₗ of the lid 128 is less than or equal to 200 microns.

In FIG. 8A, the cell deactivation container 108i is fabricated using Polydimethylsiloxane (PDMS) replication techniques. For example, the substrate 126 and channels 124 may be comprised of PDMS. In FIG. 8A, the cell deactivation container 108f is generally the same as cell deactivation container 108e except that the channels 124 are partially defined by a lamination material 130. The lamination material may be a medical grade tape or adhesive. It will be understood that the above dimensions for the thicknesses of the substrate 126, channels 124, and lid 128 may also apply to one or more other cell deactivation containers described herein.

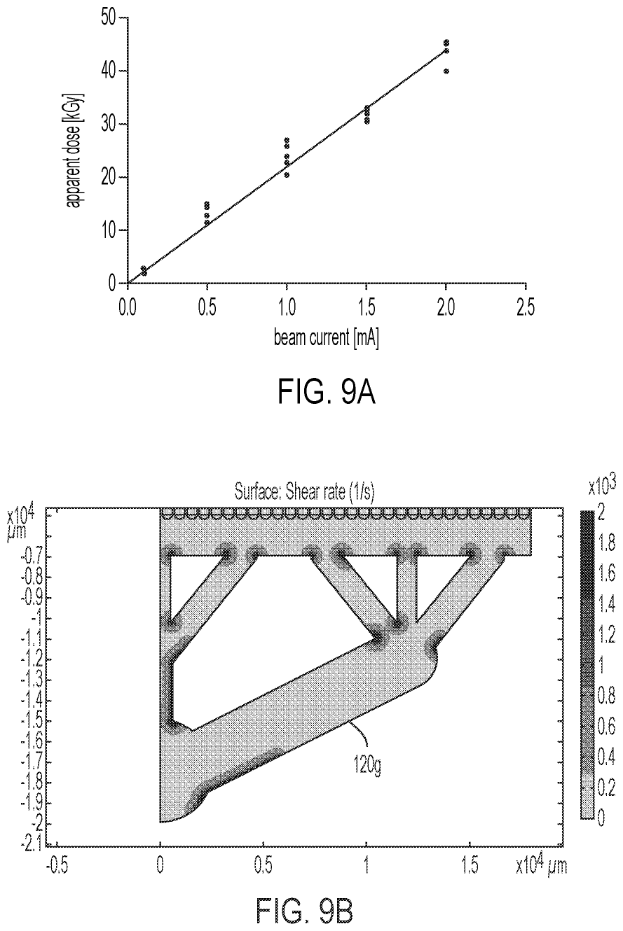

Referring to FIG. 9A there is shown a graphical representation of relation between apparent dose measured in kilograys (kGy) and beam current measured in milliamps (mA). Cell deactivation of cells contained within a liquid passed through any one of the cell deactivation containers may deactivate at about 10 Gy. Referring to FIG. 9B, there is shown a test simulation of the shear rate, measured in reciprocal seconds (l/s), of fluids passing through the first distribution layer 120g of cell deactivation container 108d. As can be seen, shear rate is relatively low and/or uniform along the majority of the first distribution layer 120g. In some embodiments, velocity of the cells may be generally independent from the electron dose. In some embodiments, as cell velocity increases, the apparent dose may decrease at a set beam current.

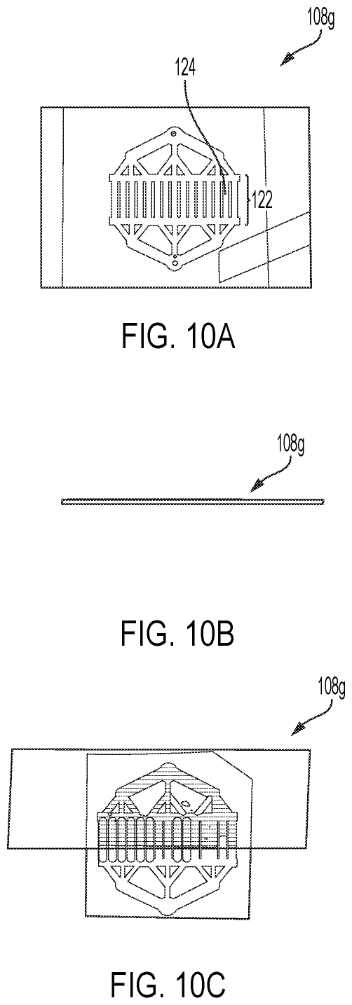

Referring to FIGS. 10A-10C, there are shown views of a cell deactivation container 108g in accordance with an exemplary embodiment of the present disclosure. The cell deactivation container 108g may be generally the same as cell deactivation container 108d except that the exposure area 122 may include channels 124 having a greater width than what is shown in FIG. 6A and the cell deactivation container 108g includes a mirrored portion that extends to the left of what is shown in FIG. 6A. In this manner, the cell deactivation containers 108a-108d may represent one half of a cell deactivation container. In some embodiments, the cell deactivation container 108g includes a glass slide, a first polymer film layer and a second polymer film layer. In some embodiments, each polymer film layer is about 100 microns thick. In some embodiments, the cell deactivation container 108g has a total penetration depth of about 200 microns. In some embodiments, the glass layer includes PDMS.

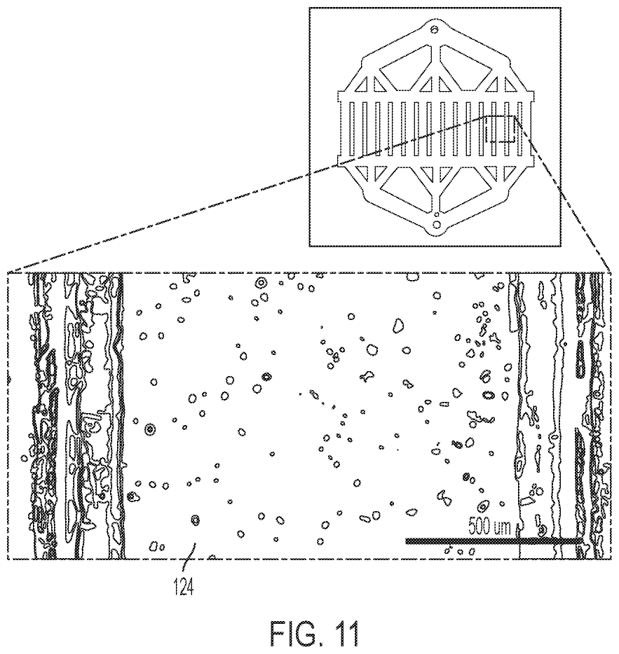

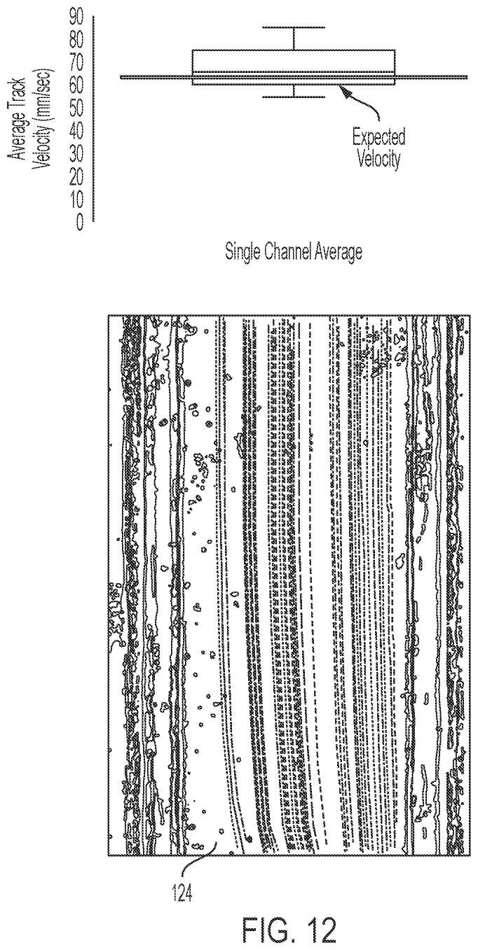

Referring to FIG. 11 there is shown a magnified view of cells within a channel 124 of the exposure layer 122 of the cell deactivation container 108g. In FIG. 11, there are SU-DHL-4 B-cells flowing through the channel 124 at a flow rate of about 500 mL/hour. Cell visualization may be accomplished by fabricating the cell deactivation container in PDMS fabrication and/or including glass. Referring to FIG. 12, there is shown a graphical representation of the measured average cell velocity of cells (e.g., SU-DHL-4 B-cells) within the channel 124 shown in FIG. 11. As shown, the measured average cell velocity was about 67.2 mm/s and the expected average velocity was 64.5 mm/s. Therefore, the average cell velocity within channel 124 is a close match to the expected average velocity.

In some embodiments, any one of the cell deactivation containers 108a-108g may be comprised of one or more materials that are stable under repeated electron beam exposure. For example, one or more of cell deactivation containers 108a-108g may include the use of one or more of a thermoset, polystyrene, a liquid crystal polymer, a polyurethane, a polyethylene, a polyester, a polycarbonate, high performance resins, silicones, PVC, polyamide (nylons), ABS, PMMA, PP (stabilized), polymethyl pentene, elastomers, cellulose, polypropylene, FEP, PTFE, and or acetals. In some embodiments, the materials may be selected based on a desired kilogray output of the electron beam. For example, an electron beam source may be provided at 65 kGy/m/min through a 0.1 mm aperture, the fluid flow rate may be 10 L/hr through a channel having a 100 micron height. In this example, the cells in the fluid are exposed to a 1.76 kGy dose and the materials of the cell deactivation container are exposed to a 180 kGy/min dose. The dose to the cell may be increased to 18 Gy thereby increasing the dose of the cell deactivation container materials to 1.8 kGy/min or 108 kGy/hr which is within the operating limits of some polymers. It will be understood that the operating limits may depend on the batch size.

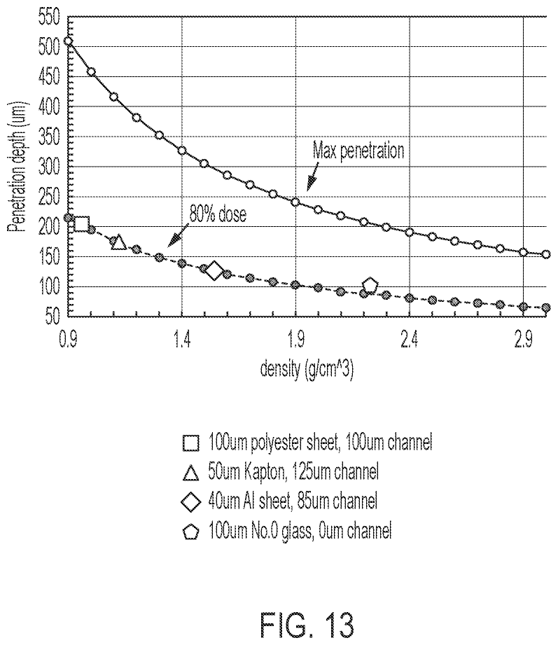

Referring to FIG. 13, there is shown a graph illustrating the penetration depth model for various two laminate materials. The upper line represents the maximum penetration and the lower line represents a minimum desired dosage percentage (e.g., 80%). In some embodiments, the minimum desired dosage percent (e.g., 80% dose) represents the strength of the electron beam at the bottom of the channel 124. For example, the minimum desired beam strength may be about 80% of the maximum beam strength at the bottom of channel 124. As such, an “effective dose” may refer to a beam strength equal to or greater than the minimum desired dosage percent at the bottom of the channel 124. Each of the square, triangle, rhombus, and pentagon shaped markers illustrated on the graph represent a different combination of lid 128 material and lid thickness with different thicknesses t_{c} of channel 124. For example, the left most marker (e.g., the square marker) represents a lid 128 comprised of polyester that has a thickness tₗ of about 100 microns. The X-axis represents the density, measured in g/cm³, of the lid 128 and/or fluid within channel 124 (e.g., water). As shown, a combination which is at or under the bottom line indicates that the two laminate material combination will receive an effective dose. For example, a lid 128 comprised of 100 micron thick polyester in combination with a 100 micron thick channel 124 results in an effective dose. Similarly, a lid 128 comprised of 50 micron thick kapton in combination with a 125 micron thick channel 124 also results in an effective dose. Furthermore, a lid 128 comprised of 40 micron thick aluminum in combination with a 85 micron thick channel 124 results in an effective dose. The combination of a lid 128 comprised of 100 micron thick No. 0 glass with a 0 micron thick channel 124 does not result in an effective does. It will be understood that although the thickness of the channel 124 is listed as zero in the combination described in the foregoing sentence, that the channel 124 may have a non-zero thickness, however, the electron beam is not capable of penetrating the lid 128 comprised of 100 micron thick glass.

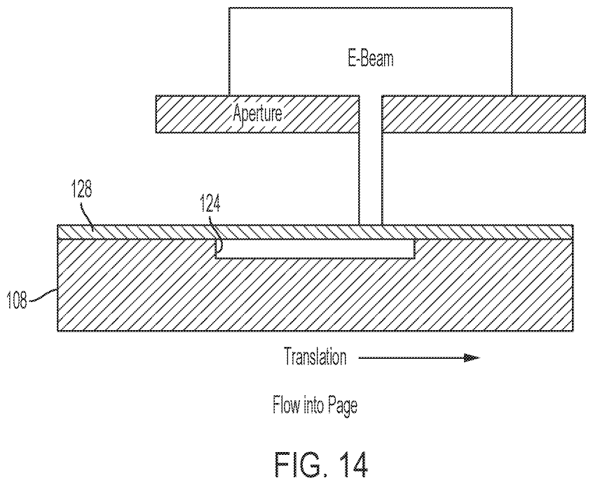

As mentioned above, continued exposure to the electron beam may have adverse effects on the materials that the cell deactivation container is comprised of. For example, plastic materials (e.g., included in the lid 128 and/or deactivation container 108) continually exposed to an electron beam may experience crosslinking and chain cleaving. Therefore, and referring to FIG. 14, the electron beam may be passed through an aperture such that the effective width of the electron beam is reduced as shown. In some embodiments, the cell deactivation container 108 may be translated in a direction generally perpendicular to the direction of the electron beam such that the exposure area of the electron beam is increased and the dose experienced by the deactivation container 108 in any one spot is decreased. Put another way, by translating the cell deactivation container 108 relative to the electron beam, no one single spot on the cell deactivation container is exposed to the electron beam for an extended period of time. It will be understood that the cell deactivation container 108 shown in FIG. 14 is an illustration and that any one of the cell deactivation containers (e.g., cell deactivation containers 108a-108g) described herein may be used with an electron beam passing through an aperture.

In some embodiments, the lid 128 may be comprised of a metal and/or plastic material configured to allow a transmitted beam of electrons to pass from the irradiation source 106 to a fluid flowing through the plurality of microfluidic channels 124. In some embodiments, providing a lid 128 comprised of a metal or metal alloy improve the cell deactivation capabilities of the deactivation container 108. For example, in some embodiments, a lid 128 comprised of a metal or metal alloy may be more resistant to any adverse effects caused by exposure to the electron beam when compared to plastic materials. Furthermore, and as illustrated in FIG. 13, lids comprised of metals or metal alloys (e.g., aluminum) may require a lesser thickness to achieve the desired minimum dosage percent when compared to plastic materials (e.g., polyester, kapton). In some embodiments, by providing a lid 128 comprised of a metal or metal alloy, a braking radiation effect of the electron beam may be enhanced. For example, the atomic nuclei of the metal or metal alloy may decelerate the electrons being transmitted through the lid 128. In this manner a lid comprised of a metal or metal alloy 128 may generate an ionizing radiation, resulting from the braking radiation, that is capable of achieving the desired minimum dosage percent (e.g., 80% dose) at lower voltages. Lower voltage electron irradiation may be safer and/or more cost effective when compared to higher voltage electron irradiation.

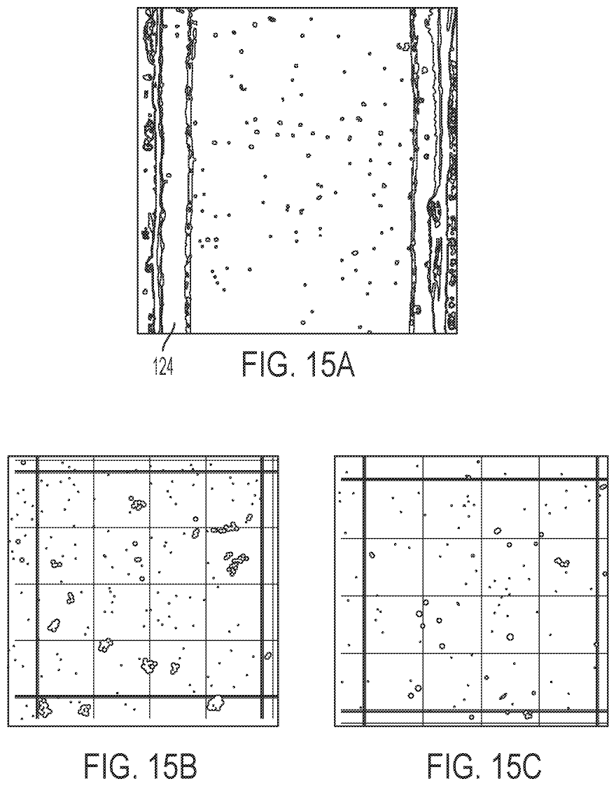

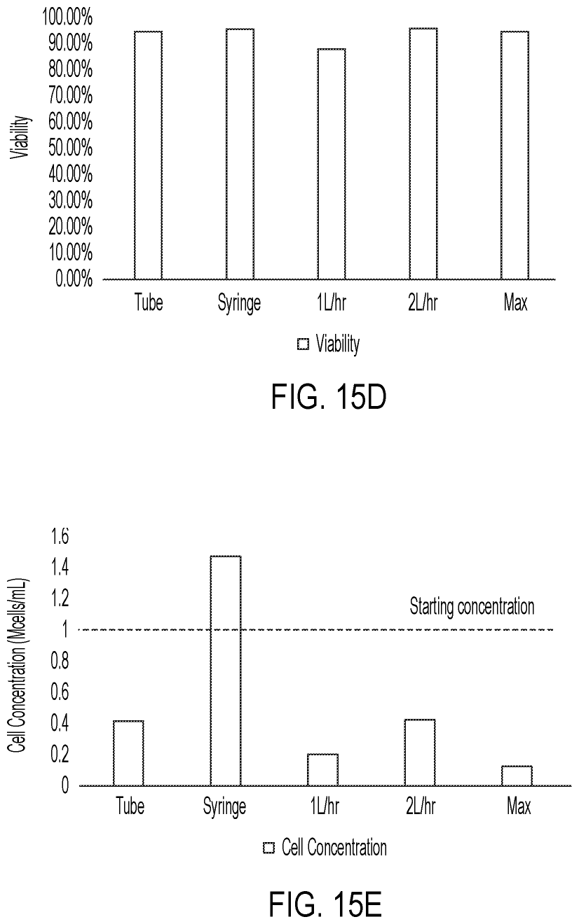

Referring to FIG. 15A, there is shown a magnified view of channel 124 containing cells included at the start of an HEK 293 cell culture during a controlled test. In the controlled test, 26×10⁶ cells were provided at a flow rate of 1×10⁶ cell/mL. The cells were processed using the microfluidic platform at 1 L/hr, 2 L/hr, and 3 L/hr. A peristaltic pump with a 25+L/hr capability was used during the testing. Viability of the samples were measured with Trypan blue 0.4% solution. There was a 1:1 ratio with cell sample and cells were counted using a hemocytometer. Referring to FIGS. 15B-15C there is shown the processing results of the HEK 293 culture from a syringe (FIG. 15B) and from the 2 L/hr sample (FIG. 15C). As shown, in the 2 L/hr sample there is low cell clumping after process. Referring to FIGS. 15D-15E there is shown bar graphs depicting the viability (FIG. 15D) and cell concentration (FIG. 15E) after processing for the various testing platforms (e.g., tube, syringe, 1 L/hr, 2 L/hr, max (e.g., 3 L/hr)) of the HEK 293 cells. As shown, there is high cell viability and cell loss between target concentration and the results of the test.

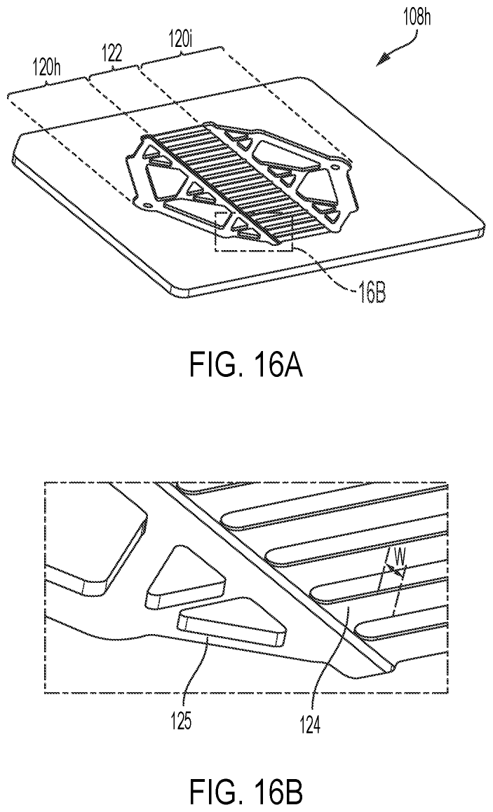

Referring to FIGS. 16A-16B, there is shown a perspective view of a cell deactivation container 108h in accordance with an embodiment of the present disclosure. The cell deactivation container 108h may be generally the same as cell deactivation container 108g except that the fluid distribution channels 125 may include one or more rounded edges where the different channels 125 branch out (e.g., where adjacent channels 125 fluidly connect to one another). For example, the distribution layers 120h and/or 120i may act as a distribution manifold configured to distribute a fluid to the plurality of microfluidic channels 124. Furthermore, the distribution layers 120h, 120i may include channels 125 having varying depths. For example, one or more of the channels 125 may have a depth which is different from one or more other channels 125. In this manner, the distribution of fluid through the channels 125 may be improved and resistance to the flow of the liquid passing through the distribution channels 120h, 120i may be reduced. In some embodiments, the channels 120h and 120i have generally the same depth.

In some embodiments, distribution layers 120h, 120i have a first depth and the exposure layer 122 has a second depth. For example, the channels 125 of the distribution layers 120h, 120i may have a first depth as measured between a bottom surface of the channels 125 and the top surface of the cell deactivation container 108 and the microfluidic channels 124 may have a second depth (e.g., thickness t_{c} in FIG. 8A-8B). In some embodiments, the first depth is greater than the second depth (e.g., the channels 125 are deeper than the exposure layer 122). In some embodiments, the depth of the exposure layer 122 (e.g., the second thickness) is about 100 microns. In some embodiments, the aspect ratio of the distribution manifold depth (e.g., the distribution layer 120h and/or distribution layer 120i) to the depth of the channels 124 is greater than or equal to about 5:1. In some embodiments, the aspect ratio of the distribution manifold depth to the depth of the channels 124 is between about 3:1 to about 50:1. In some embodiments, the aspect ratio of the distribution manifold depth to the depth of the channels 124 is at least about 7:1. In some embodiments, the aspect ratio of the distribution manifold depth to the depth of the channels 124 is at least about 10:1. In some embodiments, the aspect ratio of the distribution manifold depth to the depth of the channels 124 is at least about 15:1. In some embodiments, the aspect ratio of the distribution manifold depth to the depth of the channels 124 is at least about 25:1. In some embodiments, the aspect ratio of the distribution manifold depth to the depth of the channels 124 is less than or equal to about 25:1. In some embodiments, the thickness of the channels 125 (e.g., the first thickness) may be altered to achieve a desired flowrate. For example, the thickness of the channels 120h and 120i may be increased or decreased to alter the shear and pressure of the fluid flowing within the channels 120h and 120i.

In some embodiments, the cell deactivation container is comprised of one or more of polycarbonate (PC), poly (methyl methacrylate) (PMMA), and/or cyclic olefin copolymer (COC). Although not shown, the cell deactivation container 108h may include a lid (similar to the lid 128 shown in FIG. 8A) that is coupled to the top surface of the cell deactivation container. The lid may have a thickness less than 150 microns. In some embodiments, the thickness of the lid is about 50 microns. In some embodiments, the lid is comprised of a metal, such as, but not limited to, aluminum and/or tantalum. In some embodiments, by providing a lid comprised of a metal material, the lid may function as a better window for the electron beam to pass through when compared to lids comprised of a polymer and/or glass. Put another way, the metal lid may allow for greater penetrating radiation when compared to a lid comprised of a polymer and/or glass. Additionally, by providing a lid comprised of a metal material, the lid may be more resistant to degradation due to continued exposure from the electron beam when compared to lids comprised of a polymer and/or glass.

In some embodiments, the cell deactivation container may be configured to prevent clumping of and/or de-clump cells flowing through the exposure area 122. For example, the channels 124 of the exposure layer 122 may have a thickness that is less than the thickness of the fluid distribution channels 125 of the distribution layers 120h, 120i. In some embodiments the thickness ratio of the fluid distribution channels 125 to channels 124 is about 5:1. In some embodiments, the thickness of the channels 124 is less than 150 microns. In some embodiments, the thickness of the channels 124 is about 50 microns. In some embodiments the channels 124 each have a width W of less than 1 cm.

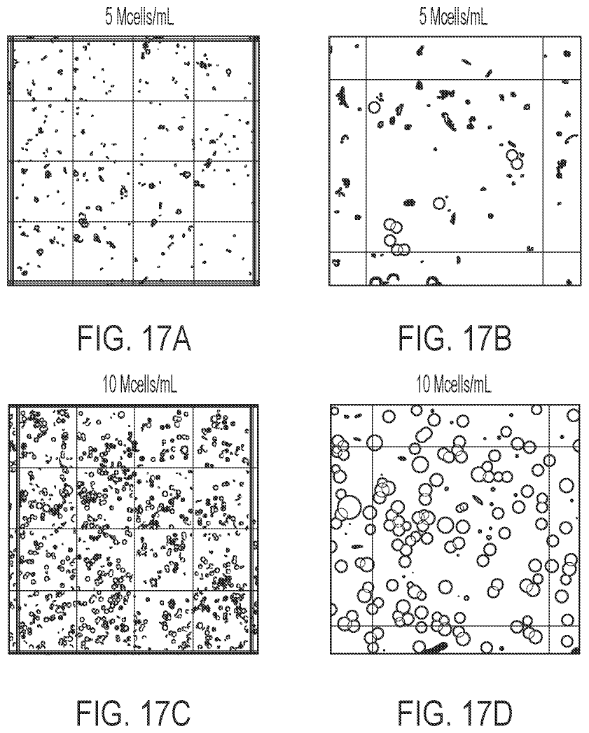

Referring to FIGS. 17A-17D there is shown results from processing high cell concentrations of HEK 293. FIG. 17A shows results from processing cell concentrations of 5×10⁶ cells/mL and FIG. 17B is a magnified view thereof. FIG. 17C shows results from processing cell concentrations of 10×10⁶ cells/mL and FIG. 17D is a magnified view thereof. Viability of the samples was tested after processing at 1 L/hr using Trypan blue staining and shows a high viability in both of the concentrations illustrated in FIGS. 17A-17D. The cells were processed without exposure to any LEEI. Concentrations of 5×10⁶ cells/mL (FIG. 17A-17B), 10×10⁶ cells/mL (FIG. 17C-17D), 15×10⁶ cells/mL (not shown), and 20×10⁶ cells/mL (not shown) were processed. The overlapping smaller images illustrate magnified views of the larger images.

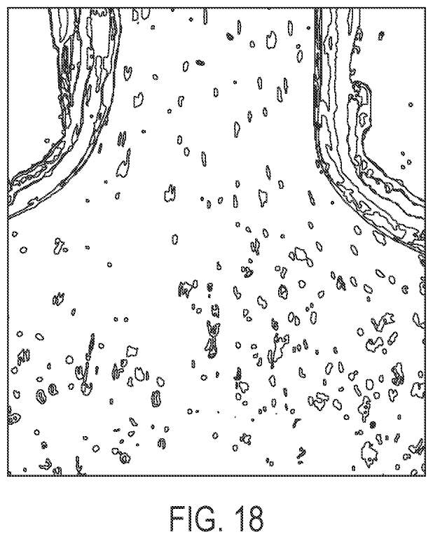

Referring to FIG. 18, there is shown an image of HEK 293 cells entering a distribution layer 120h of the cell deactivation container 108h at a concentration of 5×10⁶ cells/mL. As shown, shear at the entrance may evenly distribute the cells and disrupt clumping of cells. The image captured in FIG. 18 is taken at a concentration of 5×10⁶ cells/mL and a flow rate of 1 L/hr.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”. As used herein, the term “about” may refer to +/−10% of the value referenced. For example, “about 9” is understood to encompass 8.1 and 9.9.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

1. A cell deactivation device comprising:
a cell deactivation container comprising:
microfluidic channels, each having a maximum channel width and a maximum channel depth;
a lid covering the microfluidic channels, the lid having a lid thickness of less than about 150 microns; and
a distribution manifold configured to distribute a volumetric flow rate of a fluid to the microfluidic channels that does not vary by more than about 25% across the microfluidic channels; and
an irradiation source configured to:
generate an electron beam; and
transmit the electron beam through the lid of the cell deactivation container and into a fluid passing through the microfluidic channels;
wherein a channel aspect ratio of the maximum channel width to the maximum channel depth is less than about 10:1;
wherein a sum of the maximum channel depth and the lid thickness is no greater than about 250 microns.

2-4. (canceled)

5. The cell deactivation device of claim 1, wherein the lid thickness is between about 100 microns to about 150 microns.

6. The cell deactivation device of claim 1, wherein the lid thickness is between about 75 microns to about 125 microns.

7. The cell deactivation device of claim 1, wherein the channel aspect ratio is less than about 5:1.

8.-10. (canceled)

11. The cell deactivation device of claim 1, wherein the channel aspect ratio is from about 4:1 to about 6:1.

12. (canceled)

13. The cell deactivation device of claim 1, wherein the sum of the maximum channel depth and the lid thickness is no greater than about 200 microns.

14. The cell deactivation device of claim 1, wherein the maximum channel width is between about 0.5 millimeters to about 5.0 millimeters.

15. The cell deactivation device of claim 1, wherein the maximum channel width is between about 1.0 millimeters to about 2.0 millimeters.

16. The cell deactivation device of claim 1, wherein the lid comprises a metal and/or a plastic.

17. The cell deactivation device of claim 1, wherein the distribution manifold is configured to distribute a substantially equal volumetric flow rate of the fluid to the microfluidic channels.

18.-19. (canceled)

20. The cell deactivation device of claim 1, wherein the distribution manifold is configured to distribute the volumetric flow rate of the fluid through the microfluidic channels that does not vary by more than about 5% across the microfluidic channels.

21. The cell deactivation device of claim 1, wherein the distribution manifold is configured to distribute the volumetric flow rate of the fluid through the microfluidic channels that does not vary by more than about 1% across the microfluidic channels.

22. (canceled)

23. The cell deactivation device of claim 1, wherein the distribution manifold has a maximum distribution manifold depth; and
wherein distribution manifold/channel aspect ratio of the maximum distribution manifold depth to the maximum channel depth is between about 3:1 to about 50:1.

24.-26. (canceled)

27. The cell deactivation device of claim 23, the distribution manifold/channel aspect ratio is at least about 25:1.

28. The cell deactivation device of claim 1, wherein the electron beam has an energy of less than 100 keV.

29. A cell deactivation device comprising:
a cell deactivation container comprising:
microfluidic channels, each having a maximum channel width between about 0.5 millimeters to about 1.0 millimeters and a maximum channel depth;
a lid covering the microfluidic channels, the lid having a lid thickness of less than about 125 microns; and
a distribution manifold having a maximum distribution manifold depth and fluid distribution channels in fluid communication with one another and including one or more rounded edges where adjacent fluid distribution channels fluidly connect to one another, the distribution manifold configured to distribute a volumetric low rate of a fluid to the microfluidic channels that does not vary by more than about 5% across the microfluidic channels; and
an irradiation source configured to transmit an electron beam having an energy of less than 300 keV through the lid of the cell deactivation container and into a fluid passing through the microfluidic channels;
wherein a channel aspect ratio of the maximum channel width to the maximum channel depth of each of the microfluidic channels is less than about 5:1;
wherein a distribution manifold/channel aspect ratio of the maximum distribution manifold depth to the maximum channel depth is between about 25:1 to about 50:1;
wherein a sum of the maximum channel depth and the lid thickness is no greater than about 200 microns.

30. (canceled)

31. The cell deactivation device of claim 29, wherein the fluid one or more pathogens and/or cells; and
wherein the irradiation source and lid thickness are cooperatively configured to deactivate one or more of the pathogens and/or render one or more of the cells replication-incompetent.

32. The cell deactivation device of claim 31, wherein one or more of the pathogens is selected from the group consisting of a bacterium, a virus, a fungus, a parasite, a pathogen comprising nucleic acid molecules comprising DNA, and a pathogen comprising nucleic acid molecules comprising RNA.

33. (canceled)

34. The cell deactivation device of claim 31, wherein the deactivation of one or more of the pathogens alters gene expression of one or more of the pathogens.
